(19) United States
(12) Patent Application Publication
(10) Pub. No.: US 2003/0186400 A1
(43) Pub. Date: Oct. 2, 2003

(54) Method for producing optically active 2-hydroxycycloalkanecarboxylic acid ester

(75) Inventors: Hiroyuki Asako, Toyonaka-shi (JP); Ryuhei Wakita, Toyonaka-shi (JP); Nobuya Itoh, Toyama-shi (JP)

Correspondence Address: SUGHRUE MION, PLLC, 2100 PENNSYLVANIA AVENUE, N.W., WASHINGTON, DC, 20037, US

(73) Assignee: SUMITOMO CHEMICAL COMPANY, LIMITED

(21) Appl. No.: 10/327,108

(22) Filed: Dec. 24, 2002

(30) Foreign Application Priority Data
Dec. 27, 2001 (JP) 2001-395884
Dec. 27, 2001 (JP) 2001-395885
Apr. 10, 2002 (JP) 2002-107648

(51) Int. Cl. C12P 7/62; C12P 7/42; C12N 9/02; C07H 21/04; C12P 21/02; C12N 1/21; C12N 15/74

(52) U.S. Cl. 435/146; 435/69.1; 435/189; 435/320.1; 435/252.3; 536/23.2

(57) ABSTRACT

There is disclosed a method for producing optically active 2-hydroxycycloalkanecarboxylic acid characterized by the steps of: allowing 2-oxocycloalkanecarboxylic acid ester to react with a transformant or a dead cell artificially provided with an ability to asymmetrically reduce 2-oxocycloalkanecarboxylic acid ester to optically active 2-hydroxycycloalkanecarboxylic acid ester and an ability to regenerate a coenzyme on which an enzyme having the former ability depends; and collecting the produced optically active 2-hydroxycycloalkanecarboxylic acid ester.

### BACKGROUND OF THE INVENTION

1. Field of the Invention

The present invention relates to a method for producing optically active 2-hydroxycycloalkanecarboxylic acid esters.

Optically active 2-hydroxycycloalkanecarboxylic acid esters are useful intermediates compounds for the production of bioactive substances (for example, a compound which serves as an active ingredient of an agent having blood fat decreasing activity and antiarteriosclerosis activity, as described in Japanese Patent No.2532299), and hence an industrially advantageous production process thereof has been desired.

### SUMMARY OF THE INVENTION

According to the present invention, optically active 2-hydroxycycloalkanecarboxylic acid ester can be industrially advantageously produced.

The present invention provides:

1. a method for producing optically active 2-hydroxycycloalkanecarboxylic acid ester comprising the steps of:

(a) allowing 2-oxocycloalkanecarboxylic acid ester to react with a transformant or a dead cell thereof artificially provided with

(i) an ability to asymmetrically reduce 2-oxocycloalkanecarboxylic acid ester to optically active2-hydroxycycloalkanecarboxylic acid ester, and

(ii) an ability to regenerate a coenzyme on which an enzyme having the ability as defined in (i) depends; and

(b) collecting the resulting optically active 2-hydroxycycloalkanecarboxylic acid ester (hereinafter referred to as present production method);

2. a production method according to the item 1 above, wherein the transformant is a transformant having at least one selected from:

(A) a plasmid comprising a nucleotide sequence encoding an amino acid sequence of an enzyme having both of the two abilities (i) and (ii) as described below;

(B) a plasmid comprising

(a) a DNA having a nucleotide sequence encoding an amino acid sequence of an enzyme having an ability (i)as described below, and

(b) a DNA having a nucleotide sequence encoding an amino acid sequence of an enzyme having an ability (ii) as described below; or

(C) a pair of plasmids:

(a) a plasmid comprising a DNA having a nucleotide sequence encoding an amino acid sequence of an enzyme having an ability (i) as described below, and

(b) a plasmid comprising a DNA having a nucleotide sequence encoding an amino acid sequence of an enzyme having an ability (ii) as described below,

wherein said abilities are:

(i) an ability to asymmetrically reduce 2-oxocycloalkanecarboxylic acid ester to optically active 2-hydroxycycloalkanecarboxylic acid ester; and

(ii) an ability to regenerate a coenzyme on which an enzyme having the above ability (i) depends;

3. a production method according to the item 1 above, wherein the transformant is Escherichia coli;

4. a production method according to the item 1 above, wherein the coenzyme is NADH/NAD⁺ (nicotinamide adenine dinucleotide) or NADPH/NADP⁺ (nicotinamide adenine dinucleotide phosphate);

5. a production method according to the item 1 above, wherein the 2-oxocycloalkanecarboxylic acid ester is allowed to react with the transformant or a dead cell thereof in the presence of an aliphatic alcohol;

6. a production method according to the item 5 above, whrein the aliphatic alcohol is an alcohol having a boiling point of not more than 200° C;

7. a production method according to the item 5 above, wherein the aliphatic alcohol is 2-propanol;

8. a production method according to the item 1 above, wherein the 2-oxocycloalkanecarboxylic acid ester is allowed to react with the transformant or a dead cell thereof in the presence of glucose;

9. a production method according to the item 1 above, wherein the ability to asymmetrically reduce 2-oxocycloalkanecarboxylic acid ester to optically active 2-hydroxycycloalkanecarboxylic acid ester is an ability possessed by an enzyme having an amino acid sequence selected from the group consisting of the following amino acid sequences:

(a) an amino acid sequence represented by SEQ ID NO: 1 or 3;

(b) an amino acid sequence represented by SEQ ID NO: 1 or 3 in which one or more amino acids are deleted, substituted or added, and the amino acid sequence is an amino acid sequence of an enzyme having an ability to asymmetrically reduce 2-oxocycloalkanecarboxylic acid ester to optically active 2-hydroxycycloalkanecarboxylic acid ester;

(c) an amino acid sequence encoded by the nucleotide sequence represented by SEQ ID NO: 2 or 4;

(d) an amino acid sequence encoded by a nucleotide sequence of a DNA which hybridizes with a DNA consisting of the nucleotide sequence represented by SEQ ID NO: 2 or 4 under the stringent condition, and the amino acid sequence is an amino acid sequence of an enzyme having an ability to asymmetrically reduce 2-oxocycloalkanecarboxylic acid ester to optically active 2-hydroxycycloalkanecarboxylic acid ester; and

(e) an amino acid sequence of an enzyme

having an ability to asymmetrically reduce 2-oxocycloalkanecarboxylic acid ester to optically active 2-hydroxycycloalkanecarboxylic acid ester, and

derived from microorganism of genus Corynebacterium or genus Penicillium;

10. a production method according to the item 1 above, wherein the ability to asymmetrically reduce 2-oxocycloalkanecarboxylic acid ester to optically active 2-hydroxycycloalkanecarboxylic acid ester is an ability possessed by an enzyme having an amino acid sequence selected from the group consisting of the following amino acid sequences:

(a) an amino acid sequence represented by SEQ ID NO: 1;

(b) an amino acid sequence represented by SEQ ID NO: 1 in which one or more amino acids are deleted, substituted or added, and the amino acid sequence is an amino acid sequence of an enzyme having an ability to asymmetrically reduce 2-oxocycloalkanecarboxylic acid ester to optically active 2-hydroxycycloalkanecarboxylic acid ester;

(c) an amino acid sequence encoded by the nucleotide sequence represented by SEQ ID NO: 2;

(d) an amino acid sequence encoded by a nucleotide sequence of a DNA that hybridizes with a DNA consisting of the nucleotide sequence represented by SEQ ID NO: 2 under the stringent condition, the amino acid sequence being an amino acid sequence of an enzyme having an ability to asymmetrically reduce 2-oxocycloalkanecarboxylic acid ester to optically active 2-hydroxycycloalkanecarboxylic acid ester; and

(e) an amino acid sequence of an enzyme,

having an ability to asymmetrically reduce 2-oxocycloalkanecarboxylic acid ester to optically active 2-hydroxycycloalkanecarboxylic acid ester, and

derived from microorganism of genus Corynebacterium;

11. a production method according to the item 1 above, wherein the ability to asymmetrically reduce 2-oxocycloalkanecarboxylic acid ester to produce otptically active 2-hydroxycycloalkanecarboxylic acid ester is an ability possessed by an enzyme having an amino acid sequence selected from the group consisting of the following amino acid sequences:

(a) an amino acid sequence represented by SEQ ID NO: 3;

(b) an amino acid sequence represented by SEQ ID NO: 3 in which one or more amino acids are deleted, substituted or added, and the amino acid sequence is an amino acid sequence of an enzyme having an ability to asymmetrically reduce 2-oxocycloalkanecarboxylic acid ester to optically active 2-hydroxycycloalkanecarboxylic acid ester;

(c) an amino acid sequence encoded by the nucleotide sequence represented by SEQ ID NO: 4;

(d) an amino acid sequence encoded by a nucleotide sequence of a DNA that hybridizes with a DNA consisting of the nucleotide sequence represented by SEQ ID NO: 4 under the stringent condition, and the amino acid sequence is an amino acid sequence of an enzyme having an ability to asymmetrically reduce 2-oxocycloalkanecarboxylic acid ester to optically active 2-hydroxycycloalkanecarboxylic acid ester; and

(e) an amino acid sequence of an enzyme

having an ability to asymmetrically reduce 2-oxocycloalkanecarboxylic acid ester to optically active 2-hydroxycycloalkanecarboxylic acid ester, and

derived from microorganism of genus Penicillium;

12. a production method according to the item 1 above, wherein the ability to asymmetrically reduce 2-oxocycloalkanecarboxylic acid ester to optically active 2-hydroxycycloalkanecarboxylic acid ester is an ability possessed by an enzyme having an amino acid sequence represented by SEQ ID NO: 1;

13. a production method according to the item 1 above, wherein the ability to asymmetrically reduce 2-oxocycloalkanecarboxylic acid ester to optically active 2-hydroxycycloalkanecarboxylic acid ester is an ability possessed by an enzyme having an amino acid sequence represented by SEQ ID NO: 3;

14. a production method according to the item 1 above, wherein the ability to asymmetrically reduce 2-oxocycloalkanecarboxylic acid ester to optically active 2-hydroxycycloalkanecarboxylic acid ester is an ability possessed by an enzyme having an amino acid sequence encoded by the nucleotide sequence represented by SEQ ID NO: 2;

15. a production method according to the item 1 above, wherein the ability to asymmetrically reduce 2-oxocycloalkanecarboxylic acid ester to optically active 2-hydroxycycloalkanecarboxylic acid ester is an ability possessed by an enzyme having an amino acid sequence encoded by the nucleotide sequence represented by SEQ ID NO: 4;

16. Use of a transformant or a dead cell thereof to which an ability to asymmetrically reduce 2-oxocycloalkanecarboxylic acid ester to produce 2-hydroxycycloalkanecarboxylic acid ester and an ability to regenerate a coenzyme on which the enzyme having the former ability depends are artificially provided, as a catalyst for asymmetrically reducing 2-oxocycloalkanecarboxylic acid ester to optically active 2-hydroxycycloalkanecarboxylic acid ester.

In the following, the details of the present invention will be described.

The term “2-oxocycloalkanecarboxylic acid ester” means 2-oxocycloalkanecarboxylic acid ester compounds or salts thereof having 6 to 8 carbon atoms in the 2-oxocycloalkanecarboxylic acid moiety. Also, examples of which include, for example, 2-oxocycloalkanecarboxylic acid ester of formula (1) and a salt thereof,

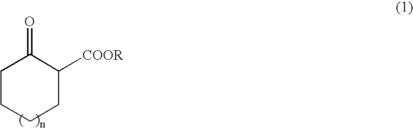

wherein, R represents an alkyl group having 1 to 8 carbon atom(s), n=0 to 2.

Examples of the C1-8 alkyl group include, for example, methyl, ethyl, n-propyl, i-propyl, n-butyl, s-butyl, i-butyl, t-butyl, n-pentyl, neo-pentyl, t-pentyl, n-hexyl, n-heptyl, n-octyl, and the like,

Specific examples of the 2-oxocycloalkanecarboxylic acid ester of formula (1) include, for example, 2-oxocyclopentanecarboxylic acid ester, 2-oxocyclohexanecarboxylic acid ester, and 2-oxocycloheptanecarboxylic acid ester. The term “2-hydroxycycloalkanecarboxylic acid ester” means hydroxyl compounds or salts thereof which can be obtained by reducing a keto group of the aforementioned 2-oxocycloalkanecarboxylic acid ester. More specifically, when 2-oxocycloalkanecarboxylic acid ester of formula (1) is used as a material compound in the present production method, optically active 2-hydroxycycloalkanecarboxylic acid ester, which is a hydroxyl compound corresponding thereto, of formula (2):

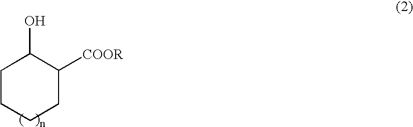

wherein, R represents an alkyl group having 1 to 8 carbon atoms, n=0 to 2, the carbon atom to which the hydroxy group is bonded is an asymmetric carbon atom, is produced.

Specific examples of the optically active 2-hydroxycycloalkanecarboxylic acid ester of formula (2) include, for example, optically active:

methyl 2-hydroxycyclopentanecarboxylate,

ethyl 2-hydroxycyclopentanecarboxylate,

methyl 2-hydroxycyclohexanecarboxylate,

ethyl 2-hydroxycyclohexanecarboxylate,

methyl 2-hydroxycycloheptanecarboxylate,

ethyl 2-hydroxycycloheptanecarboxylate, and

propyl, butyl, pentyl, hexyl or octyl esters of the above-described esters.

The transformant suitably used in the production method according to the present invention is a transformant to which an ability to asymmetrically reduce 2-oxocycloalkanecarboxylic acid ester to optically active 2-hydroxycycloalkanecarboxylic acid ester and an ability to regenerate a coenzyme on which an enzyme having the former ability depends are artificially imparted.

Examples of the transformant include, for example, a transformant having at least one selected from:

(A) a plasmid comprising a nucleotide sequence encoding an amino acid sequence of an enzyme having both of the two abilities as described below;

(B) a plasmid comprising

(a) a DNA having a nucleotide sequence encoding an amino acid

sequence of an enzyme having an ability (i) as described below, and

(b) a DNA having a nucleotide sequence encoding an amino acid sequence of an enzyme having an ability (ii) as described below; or

(C) a pair of plasmids:

(a) a plasmid comprising a DNA having a nucleotide sequence encoding an amino acid sequence of an enzyme having an ability (i) as described below, and

(b) a plasmid comprising a DNA having a nucleotide sequence encoding an amino acid sequence of an enzyme having an ability (ii) as described below,

wherein said abilities are:

(i) an ability to asymmetrically reduce 2-oxocycloalkanecarboxylic acid ester to optically active 2-hydroxycycloalkanecarboxylic acid ester; and

(ii) an ability to regenerate a coenzyme on which an enzyme having the above ability (i) depends.

Such a transformant usually contains (1) an enzyme having both of the two abilities that are artificially imparted, i.e., the ability to asymmetrically reduce 2-oxocycloalkanecarboxylic acid ester to optically active 2-hydroxycycloalkanecarboxylic acid ester and the ability to regenerate a coenzyme on which the enzyme having the former ability depends, or (2) two kinds of enzymes, i.e., an enzyme having an ability to asymmetrically reduce 2-oxocycloalkanecarboxylic acid ester to optically active 2-hydroxycycloalkanecarboxylic acid ester and an enzyme having an ability to regenerate a coenzyme on which the enzyme having the former ability depends (hereinafter, the enzymes (1) and (2) are often referred to as generally “present enzyme”).

Further, concrete examples of “the ability to asymmetrically reduce 2-oxocycloalkanecarboxylic acid ester to optically active 2-hydroxycycloalkanecarboxylic acid ester” include abilities possessed by the enzyme(s) having an amino acid sequence, for example, selected from the group of amino acid sequences as follows:

(a1) an amino acid sequence represented by SEQ ID NO: 1,

(a2) an amino acid sequence represented by SEQ ID NO: 3,

(b1) an amino acid sequence represented by SEQ ID NO: 1 in which one or more amino acids are deleted, substituted or added, and the amino acid sequence is an amino acid sequence of an enzyme having an ability to asymmetrically reduce 2-oxocycloalkanecarboxylic acid ester to optically active 2-hydroxycycloalkanecarboxylic acid ester,

(b2) an amino acid sequence represented by SEQ ID NO: 3 in which one or more amino acids are deleted, substituted or added, and the amino acid sequence is an amino acid sequence of an enzyme having an ability to asymmetrically reduce 2-oxocycloalkanecarboxylic acid ester to optically active 2-hydroxycycloalkanecarboxylic acid ester,

(c1) an amino acid sequence encoded by the nucleotide sequence represented by SEQ ID NO: 2,

(c2) an amino acid sequence encoded by the nucleotide sequence represented by SEQ ID NO: 4,

(d1) an amino acid sequence encoded by a nucleotide sequence of a DNA that hybridizes with a DNA consisting of the nucleotide sequence represented by SEQ ID NO: 2 under the stringent condition, and the amino acid sequence is an amino acid sequence of an enzyme having an ability to asymmetrically reduce 2-oxocycloalkanecarboxylic acid ester to optically active 2-hydroxycycloalkanecarboxylic acid ester,

(d2) an amino acid sequence encoded by a nucleotide sequence of a DNA that hybridizes with a DNA consisting of the nucleotide sequence represented by SEQ ID NO: 4 under the stringent condition, and the amino acid sequence is an amino acid sequence of an enzyme having an ability to asymmetrically reduce 2-oxocycloalkanecarboxylic acid ester to optically active 2-hydroxycycloalkanecarboxylic acid ester,

(e1) an amino acid sequence of an enzyme,

having an ability to asymmetrically reduce 2-oxocycloalkanecarboxylic acid ester to optically active 2-hydroxycycloalkanecarboxylic acid ester, and

derived from microorganism of genus Corynebacterium, and

(e2) an amino acid sequence of an enzyme, having an ability to asymmetrically reduce 2-oxocycloalkanecarboxylic acid ester to optically active 2-hydroxycycloalkane- carboxylic acid ester, and

derived from microorganism of genus Penicillium.

The present transformant may be produced by using genetic engineering technique. It is to be noted that a gene (hereinafter, also referred to as “present reductase gene”) including a nucleotide sequence encoding an amino acid sequence of an enzyme (hereinafter, also referred to as “present reductase”) having an ability to asymmetrically reduce 2-oxocycloalkanecarboxylic acid ester to optically active 2-hydroxycycloalkanecarboxylic acid ester for use in generating such a transformant may be obtained from (1) naturally occurring genes by cloning technique, (2) a gene obtained from naturally occurring genes by cloning technique in which a part of nucleotides may be artificially deleted, substituted or added (that is, naturally occurring gene on which a mutation process (partial mutation introducing method, mutagenesis treatment and the like) is effected) or (3) artificially synthesized.

In this context, the terms “amino acid sequence in which one or more amino acids are deleted, substituted or added” as described in the aforementioned (b), (b1) or (b2) and “amino acid sequence encoded by a nucleotide sequence of a DNA which hybridizes under the stringent condition” described in the aforementioned (d), (d1) or (d2) include processings that the enzyme having the amino acid sequence represented by SEQ ID NO: 1 or SEQ ID NO: 3 experiences in a cell, mutations naturally occurring due to the individual difference, species difference and difference between different tissues of the organism from which the enzyme originates, and artificial mutations of one or more amino acids.

As a procedure for artificially achieving “deletion, substitution or addition of one or more amino acids ” (hereinafter, also referred to as “modification of amino acid”) described in the aforementioned (b), (b1) or (b2), there is a procedure which includes subjecting a DNA having a nucleotide sequence encoding the amino acid sequence, for example, represented by SEQ ID NO: 1 or SEQ ID NO: 3 to the conventional site-directed mutagenesis followed by expression of the DNA by means of a well-known method. Examples of the site-directed mutagenesis include, for example, a method using amber mutation (gapped-duplex method, Nucleic Acids Res., 12, 9441-9456 (1984)) and a method based on the PCR with the use of a primer for introducing mutation.

The number of amino acids to be modified in the above is at least one residue, more specifically one or several, or more residue(s). This number of modification may be any number within which the ability to asymmetrically reduce 2-oxocycloalkanecarboxylic acid ester to optically active 2-hydroxycycloalkanecarboxylic acid ester is not impaired.

Furthermore, it is preferred that the modification is particularly substitution of amino acid among the deletion, substitution and addition as recited above. More preferably, the substitution is effected on amino acids having similar properties such as hydrophobicity, charge, pK, and features in conformation. As such a substitution, for example, substitutions between residues within the following 6 respective groups: 1. glycine, alanine; 2. valine, isoleucine, leucine; 3. aspartic acid, glutamic acid, asparagine, glutamine; 4. serine, threonine; 5. lysine, arginine; and 6. phenylalanine, tyrosine.

In the present invention, “one or more amino acids are deleted, substituted or added” means that deletion, substitution, or addition of one or more amino acids in an amino acid sequence is conducted without impairing a high sequence homology concerning amino acid sequence between the amino acid sequence and the resulting amino acid sequence by such deletion, substitution or additon therefrom, and the high sequence homology is, for example, a homology of 80% or more, preferably 90% or more, more preferably 95% or more. Furthermore, “hybridizing under the stringent condition” means that there is a sequence homology concerning nucleotide sequence between two hybridizing DNAs under the said hybridizing condition, and such sequence hology is, for example, a homology of 80% or more, preferably 90% or more, more preferably 95% or more.

In this context, the term “sequence homology” means that sequences of two DNAs or two proteins are equivalent or homologous. The aforementioned “sequence homology” is determined by comparing two sequences aligned in the optimum condition over the sequences to be compared. The two DNAs or proteins to be compared may have an addition or deletion (for example, gap) in the optimum alignment of two sequences. Such a sequence homology can be calculated by creating an alignment with the use of, for example, Vector NTI while utilizing ClustalW algorism (Nucleic Acid Res. 22(22): 4673-4680(1994)). A sequence homology is measured by using sequence analysis software, more specifically, Vector NTI, GENETYX-MAC or analysis tools provided by public databases. The public databases are commonly available, for example, in the URL address of http://www.ddbj.nig.ac.jp).

As for “which hybridizes under the stringent condition” as described in the aforementioned (d), (d1) and (d2), hybridization used herein can be conducted in accordance with common methods such as a method described in “Molecular Cloning 2nd edition (Cold Spring Harbor Laboratory press)” written by Sambrook J., Frisch E. F., Maniatis T. or Southern hybridization method described in “Cloning and Sequence” (NOSONBUNKASHA, 1989) edited by MASAHIRO SUGIURA under the editorship of ITARU WATANABE. Furthermore, examples of the “stringent condition” include such a condition that after allowing hybridization at 65° C. in a solution containing 6×SSC (a solution containing 900 mM NaCl and 90 mM sodium citrate: in this case, a solution containing 175.3 g of NaCl and 88.2 g sodium citrate is dissolved in 800 mL of water, and filled up to the total volume of 1000 mL after adjustment of pH by 10N NaCl, which is defined as 20×SSC), the resultant solution is washed with 2×SSC at 50° C. (Molecular Biology, John Wiley & Sons, N.Y. (1989), 6.3.1-6.3.6.) The salt concentration in the washing step may be selected from, for example, between the condition of 2×SSC at 50° C. (low stringency condition) and the condition of 0.1×SSC at 65° C. (high stringency condition.) The temperature in the washing step may be selected from, for example, between the room temperature (low stringency condition) and 65° C. (high stringency condition.) Also, both the salt concentration and the temperature may be varied.

The present reductase gene may be prepared, for example, in accordance with the preparation method as follows.

First a chromosomal DNA is prepared from microorganisms belonging to genus Corynebacterium such as Corynebacterium pseudodiphteriticum in accordance with a commonly-used genetic engineering technique. Then by conducting a suitable PCR reaction while using the chromosomal DNA thus prepared as a template and using a suitable primer, a DNA having a nucleotide sequence encoding the amino acid sequence represented by SEQ ID NO: 1, a DNA having a nucleotide sequence encoding the amino acid sequence represented by SEQ ID NO: 1 in which one or more amino acids are deleted, substituted or added, a DNA having a nucleotide sequence represented by SEQ ID NO: 2 or the like is amplified, whereby the present reductase gene is prepared.

In the above, when the PCR is conducted while using a chromosomal DNA derived from Corynebacterium pseudodiphteriticum as a template and an oligonucleotide having a nucleotide sequence represented by SEQ ID NO: 5 and an oligonucleotide having a nucleotide sequence represented by SEQ ID NO: 6 as primers, a DNA having a nucleotide sequence represented by SEQ ID NO: 2 is amplified, whereby the present reductase gene is prepared.

The condition for the above PCR may be such a condition that includes mixing each 20 μM of four kinds of dNTPs, each 15 pmol of two kinds of oligonucleotide primers, 1.3 U of Taq polymerase and a DNA library which serves as a template, heating the resultant mixture at 97° C. (2 min.), then repeating a cycle consisting of 97° C. (0.25 min.), 50° C. (0.5 min.) and 72° C. (1.5 min.) for 10 times, repeating a cycle consisting of 97° C. (0.25 min.), 55° C. (0.5 min.) and 72° C. (2.5 min.) for 20 times, and holding the mixture for 7 min. at 72° C.

At the 5′ ends of the primers to be used in the above PCR, sequences which are recognizable by restriction enzymes may be added.

The DNA amplified in the manner as described above can be cloned into a vector in accordance with a methodology as described in Sambrook J., Frisch E. F., Maniatis T. “Molecular Cloning: A Laboratory Manual 2nd edition” (1989), Cold Spring Harbor Laboratory Press, “Current Protocols in Molecular Biology” (1987), John Wiley & Sons, Inc.ISBNO-471-50338-X and the like to thereby obtain a recombinant vector. Concrete examples of the vector to be used include pUCl19 (available from TAKARA SYUZO CO., LTD.), pTV118N (available from TAKARA SYUZO CO., LTD.), pBluescriptII (available from TOYOBO CO., LTD.), pCR2.1-TOPO (available from Invitrogen Corporation), pTrc99A (available from Pharmacia Corporation), pKK223-3 (available from Pharmacia Corporation) and the like. The present reductase gene thus prepared in the form of being incorporated in the vector will be convenient for use in the subsequent genetic engineering methodology.

On the other hand, a cDNA library is prepared from microorganisms belonging to genus Penicillium such as (Penicillium citrinum) in accordance with a commonly-used genetic engineering methodology (for example, a method described in “New Cell Engineering Laboratory Protocol (edited by Department of Oncology, Institute of Medical Science, Univ. of Tokyo, SYUJUNSHA, 1993)), and a PCR using the prepared cDNA library as a template as well as using suitable primers is performed for amplifying a DNA having a nucleotide sequence encoding the amino acid sequence represented by SEQ ID NO: 3, a DNA having a nucleotide sequence encoding the amino acid sequence represented by SEQ ID NO: 3 in which one or more amino acid(s) is deleted, substituted or added, a DNA having a nucleotide sequence represented by SEQ ID NO: 4 or the like, whereby the present reductase gene is prepared.

In the above, when the PCR is performed while using a cDNA derived from Penicillium citrinum as a template and an oligonucleotide having a nucleotide sequence represented by SEQ ID NO: 7 and an oligonucleotide having a nucleotide sequence represented by SEQ ID NO: 8 as primers, a DNA having a nucleotide sequence represented by SEQ ID NO: 4 is amplified, whereby the present reductase gene is prepared.

The condition for the above PCR may be such a condition that includes mixing each 20 μM of four kinds of dNTPs, each 15 pmol of two kinds of oligonucleotide primers, 1.3 U of Taq polymerase and a cDNA library which serves as a template, heating the resultant mixture at 97° C. (2 min.), then repeating a cycle consisting of 97° C. (0.25 min.), 50° C. (0.5 min.) and 72° C. (1.5 min.) for 10 times, repeating a cycle consisting of 97° C. (0.25 min.), 55° C. (0.5 min.) and 72° C. (2.5 min.) for 20 times, and holding the mixture for 7 min. at 72° C.

At the 5′ ends of the primers to be used in the above PCR, sequences which are recognizable by restriction enzymes may be added.

The DNA amplified in the manner as described above can be cloned into a vector in accordance with a methodology as described in Sambrook J., Frisch E. F., Maniatis T. “Molecular Cloning: A Laboratory Manual 2nd edition” (1989), Cold Spring Harbor Laboratory Press, “Current Protocols in Molecular Biology” (1987), John Wiley & Sons, Inc. ISBNO-471-50338-X and the like to thereby obtain a recombinant vector. Concrete examples of the vector for use include pUCl19 (available from TAKARA SYUZO CO., LTD.), pTVl18N (available from TAKARA SYUZO CO., LTD.), pBluescriptII (available from TOYOBO CO., LTD.), pCR2.1-TOPO (available from Invitrogen Corporation), pTrc99A (available from Pharmacia Corporation), pKK223-3 (available from Pharmacia Corporation) and the like. The present reductase gene thus prepared in the form of being incorporated in the vector will be convenient for use in the subsequent genetic engineering methodology.

The enzyme having an ability to regenerate a coenzyme on which an enzyme having an ability to asymmetrically reduce 2-oxocycloalkanecarboxylic acid ester to produce 2-hydroxycycloalkanecarboxylic acid ester depends (hereinafter, also referred to as “present coenzyme regenerating enzyme gene”) may be prepared in the preparation method as follows, for example, when the present coenzyme regenerating enzyme gene differs from the present reductase gene.

A chromosomal DNA is prepared from microorganisms belonging to genus Bacillus such as Bacillus megaterium in accordance with a commonly-used genetic engineering technique, and then PCR is conducted while using the chromosomal DNA thus prepared as a template and a suitable primer, whereby a DNA having a nucleotide sequence encoding the amino acid sequence represented by SEQ ID NO: 12, a DNA having a nucleotide sequence encoding the amino acid sequence represented by SEQ ID NO: 12 in which one or more amino acid(s) is deleted, substituted or added, a DNA having a nucleotide sequence represented by SEQ ID NO: 11 or the like is amplified, thereby preparing the present coenzyme regenerating enzyme gene.

In the above, when the PCR is conducted while using a chromosomal DNA derived from Bacillus megaterium as a template and an oligonucleotide having a nucleotide sequence represented by SEQ ID NO: 10 and an oligonucleotide having a nucleotide sequence represented by SEQ ID NO: 10 as primers, a DNA having a nucleotide sequence represented by SEQ ID NO: 11 is amplified, whereby the present coenzyme regenerating enzyme gene is prepared.

The condition for the above PCR may be such a condition that includes mixing each 20 μM of four kinds of dNTPs, each 15 pmol of two kinds of oligonucleotide primers, 1.3 U of Taq polymerase and a DNA library which serves as a template, heating the resultant mixture at 97° C. (2 min.), then repeating a cycle consisting of 97° C. (0.25 min.), 50° C. (0.5 min.) and 72° C. (1.5 min.) for 10 times, repeating a cycle consisting of 97° C. (0.25 min.), 55° C. (0.5 min.) and 72° C. (2.5 min.) for 20 times, and holding the mixture for 7 min. at 72° C.

At the 5′ ends of the primers to be used in the above PCR, sequences which are recognizable by restriction enzymes may be added.

The DNA amplified in the manner as described above can be cloned into a vector in accordance with a methodology as described in Sambrook J., Frisch E. F., Maniatis T. “Molecular Cloning: A Laboratory Manual 2nd edition” (1989), Cold Spring Harbor Laboratory Press, “Current Protocols in Molecular Biology” (1987), John Wiley & Sons, Inc. ISBNO-471-50338-X and the like to thereby obtain a recombinant vector. Concrete examples of the vector for use include pUCl19 (available from TAKARA SYUZO CO., LTD.), pTVl18N (available from TAKARA SYUZO CO., LTD.), pBluescriptII (available from TOYOBO CO., LTD.), pCR2.1-TOPO (available from Invitrogen Corporation), pTrc99A (available from Pharmacia Corporation), pKK223-3 (available from Pharmacia Corporation) and the like. The present coenzyme regenerating gene thus prepared in the form of being incorporated in the vector will be convenient for use in the subsequent genetic engineering methodology.

As the method for preparing the present transformant, the following methods can be exemplified.

(1) A preparation method including the steps of preparing a recombinant plasmid capable of expressing the present genes in a host cell, the recombinant plasmid being such a DNA wherein both of the present reductase gene and the coenzyme regenerating enzyme gene are operably linked with a promoter which is operable in the host cell; and introducing the recombinant plasmid into the host cell.

(2) A preparation method including the steps of separately preparing two recombinant plasmids each capable of expressing either one of the present genes in a host cell, each recombinant plasmid being such a DNA wherein either one of the present reductase gene or the coenzyme regenerating enzyme gene is operably linked with a promoter which is operable in the host cell; and introducing these recombinant plasmids into the host cell. Furthermore, a method wherein either one or both of the genes are introduced in a chromosome of a host cell is also available.

As the above method for constructing the present transformant by introducing a recombinant plasmid into a host cell, the following methods can be exemplified: a method of constructing a recombinant plasmid by coupling the regions responsible for expression control such as promoter and terminator to the respective genes; and a method of constructing a recombinant plasmid so that the recombinant plasmid will be expressed as an operon including a plurality of cistrons such as lactose operon.

As the recombinant plasmid as described above, plasmids wherein a gene encoding the present enzyme is introduced in an operable form into an expression vector including genetic information which can be duplicated in a host cell, capable of replication, as well as readily isolated and purified from the host cell and having a promoter which is operable in the host cell and a detectable marker can be preferably exemplified. Various types of expression vectors are commercially available.

Herein, the terms “in the operable form” means that when a host cell is transformed by introducing the above recombinant plasmid into the host cell, the present genes (or either one of the present genes) are coupled to the promoter so that it will be expressed under the control of the promoter. Examples of the promoter include a promoter of lactose operon derived from E. coli, tryptophan operon derived from E. coli, or synthetic promoters which are operable in E. coli, such as tac promoter and trc promoter. Also, promoters that control expression of the present genes in Corynebacterium pseudodiphteriticum, Penicillium citrinum and Bacillus megaterium may be used.

Furthermore, by using a vector including a selective marker (for example, antibiotic resistance providing genes such as kanamycin resistant gene, neomycine resistant gene) as an expression vector, it is possible to readily select a transformant into which the objective vector has been introduced from the phenotype of the selective marker which serves as the index.

In the case where higher expression is required to be induced, a ribosome biding site may be coupled upstream the gene having a nucleotide sequence encoding an amino acid sequence of the present reductase and/or the present coenzyme regenerating enzyme. Examples of ribosome biding site for use include those described in the reports by Guarente L. et al. (Cell 20, p543) and Taniguchi et al. (Genetics of Industrial Microorganisms, p202, KODANSHA.)

As the host cell, microorganism cells including prokaryotes (for example, genus Eseherichia, genus Bacillus, genus Corynebacterium, genus Staphylococcus and genus Streptomyces) or eukaryotes (for example, genus Saccharomyces, genus Kluyveromyces and genus Aspergillus), insect cells or mammalian cells can be exemplified. For example, from the view point of enabling mass production of the present transformant, Escherichia coli are preferred.

As the method for introducing a plasmid capable of expressing the present reductase and/or the present coenzyme regenerating enzyme in the host cell, any introduction methods which are commonly used for the host cell are available, and examples of which include the calcium chloride method as described, for example, in “Molecular Cloning: A Laboratory Manual 2nd edition” (1989), Cold Spring Harbor Laboratory Press, “Current protocols in Molecular Biology” (1987), John Wiley & Sons, Inc. ISBNO-471-50338-X, and the electroporation described, for example, in “Methods in Electroporation: Gene Pulser/E.coli Pulser System” Bio-Rad Laboratories, (1993).

For selecting a transformant into which a plasmid capable of expressing the present reductase and/or the present coenzyme regenerating enzyme in a host cell has been introduced, the selection may be achieved while regarding the phenotype of the selective marker gene contained in the vector as an index, for example, as described above.

The fact that the host cell into which a plasmid has been introduced (namely, transformant) possesses the present reductase gene and the present coenzyme regenerating enzyme can be confirmed by performing identification of the restriction site, analysis of nucleotide sequence, Southern hybridization, Western hybridization and the like in accordance with the commonly-used methods as described in “Molecular Cloning: A Laboratory Manual 2nd edition” (1989), Cold Spring Harbor Laboratory press and the like.

Cultivation of the present transformant is performed in accordance with methods which are commonly used for cultivation of microorganisms, or cultivation of insect cells or mammalian cells. For instance, in the case of Escherichia coli, cultivation is performed in a culture medium containing suitable carbon source, nitrogen source, and trace nutrients such as vitamins as necessary. As the cultivation method, any of solid culture, liquid culture such as test tube shaking culture, reciprocative shaking culture and Jar Fermenter culture and tank culture is available, and preferably liquid culture such as aeration and agitation culture can be recited.

The cultivation temperature is generally about 10 to 50° C., preferably about 20 to 40° C., though it can be appropriately varied within the range of the growth temperature of the present transformant. The pH of the culture medium is preferably about 6 to 8. Preferred cultivation time is usually about 1 day to about 5 days though it differs in accordance with the cultivation condition.

As the culture medium for cultivating the present transformant, a variety of culture media which appropriately containing a carbon source, nitrogen source, as well as organic salts, inorganic salts and the like which are commonly used in cultivation of host cells such as microorganisms can be used, for example.

Examples of the carbon source include saccharides such as glucose, dextrin and sucrose, sugar alcohols such as glycerol, organic acids such as fumaric acid, citric acid and pyruvic acid, animal oils, vegetable oils and molasses. The adding amount of these carbon sources to the culture medium is usually about 0.1 to 30% (w/v) with respect to the culture.

Examples of the nitrogen source include natural organic nitrogen sources such as meat extract, peptone, yeast extract, malt extract, soy bean powder, Corn Steep Liquor, cotton seed powder, dry yeast and casamino acids; amino acids; sodium salts of inorganic acids such as sodium nitrate; ammonium salts of inorganic acids such as ammonium chloride, ammonium sulfate and ammonium phosphate; ammonium salts of organic acids such as ammonium fumarate and ammonium citrate; and urea. Among these, ammonium salts of organic acids, natural organic nitrogen sources, amino acids and the like are often used as a carbon source. The adding amount of these nitrogen sources to the culture medium is usually about 0.1 to 30% (w/v) with respect to the culture.

Examples of the organic salts and inorganic salts include chlorides, sulfates, acetates, carbonates and phosphates of potassium, sodium, magnesium, iron, manganese, cobalt, zinc and the like. Concrete examples include sodium chloride, potassium chloride, magnesium sulfate, ferrous sulfate, manganese sulfate, cobalt chloride, zinc sulfate, copper sulfate, sodium acetate, calcium carbonate, potassium dihydrogenphosphate and dipotassium hydrogenphosphate. The adding amount of these organic salts and inorganic salts to the culture medium is usually about 0.0001 to 5% (w/v) with respect to the culture.

Furthermore, in the case of a transformant transformed with a DNA wherein a promoter of the allolactose-inducible type such as tac promoter, trc promoter and lac promoter is coupled with a gene having a nucleotide sequence encoding an amino acid sequence of the present enzyme in an operable manner, for example, a small amount of isopropyl thio-β-D-galactoside (IPTG) may be added to the culture medium as an inducing agent for inducing production of the present enzyme.

The present transformant can be obtained by collecting the transformant in the form of a precipitate, for example, by centrifugal separation of the culture obtainable in the aforementioned cultivation. Prior to the collection, the transformant may be appropriately washed, for example, with a buffer such as 100 mM potassium dihydrogenphosphate/dipotassium hydrogenphosphate buffer (pH 6.5).

Moreover, a dead cell may be prepared from the present transformant in the manner as described below.

As the method for preparing a dead cell, physically sterilizing methods (e.g. heating, drying, freezing, ultrasonic wave, filtration,) and sterilizing methods using chemicals (alkalis, acids, halogens, oxidants, sulfur, boron, arsenic, metals, alcohols, phenols, amines, sulfides, ethers, aldehydes, ketones, cyans and antibiotics) can be exemplified. It is desirable to select a treating method which inactivates the enzyme activity of the present enzyme as minimum as possible, and is unlikely to cause a residence and contamination on the reaction system appropriately in accordance with a variety of reaction conditions.

The transformant or its dead cell thus prepared may be used, for example, in the form of lyophilized cell, organic solvent treated cell, dried cell and the like, or in an immobilized form (immobilized substance).

Examples of the method for obtaining an immobilized substance include carrier biding methods (the present transformant or dead cell thereof is adsorbed to an inorganic carrier such as silica gel or ceramic, cellulose, ion-exchanged resin, and the like), and entrapment (the present transformant or dead cell thereof is entrapped into a mesh structure of polymer such as polyacrylamide, sulfur-containing polysaccharide gel (e.g., carrageenan gel), alginic acid gel, agar gel and the like).

Next, a catalytic reaction in the present production method will be explained.

In the present production method, the reaction for converting 2-oxocycloalkanecarboxylic acid ester into optically active 2-hydroxycycloalkanecarboxylic acid ester is achieved by allowing the present transformant or dead cell thereof to react with 2-oxocycloalkanecarboxylic acid ester.

The above reaction is carried out in the presence of water. The water may be in the form of a buffer, and examples of such buffer include phosphates of alkali metals such as sodium phosphate and potassium phosphate, and acetates of alkali metals such as sodium acetate and potassium acetate.

When a buffer is used as a solvent, the amount of the buffer is generally 1 to 300 times by weight, preferably 5 to 100 times by weight with respect to 1 part by weight of 2-oxocycloalkanecarboxylic acid ester.

In the above reaction, 2-oxocycloalkanecarboxylic acid ester may be added to the reaction system continuously or occasionally.

The reaction temperature can be about 0 to 70° C. from the view points of the stability and the reaction rate of the present enzyme contained in the present transformant or a dead cell thereof, and preferably about 10 to 40° C.

The reaction pH is usually, for example, 5 to 8, although it can be appropriately varied within the range that allows process of the reaction.

The reaction may be carried out in the presence of an organic solvent besides water. Examples of such organic solvent include ethers such as tetrahydrofuran, t-butylmethylether and isopropylether, hydrocarbons such as toluene, hexane, cyclohexane, heptane, isooctane and decane, alcohols such as t-butanol, methanol, ethanol, isopropanol and n-butanol, sulfoxides such as dimethylsulfoxide, ketones such as acetone, nitrites such as acetonitrile and mixtures thereof.

The amount of the organic solvent that may be suitably used for the reaction is usually not more than 100 times by weight, preferably not more than 70 times by weight with respect to 2-oxocycloalkanecarboxylic acid ester.

Usually, it is preferred that the reaction is carried out while adding a coenzyme such as NADH and NADPH, for example.

The amount of coenzyme that may be used for the reaction is usually not more than 0.5 times by weight, preferably not more than 0.1 times by weight with respect to 2-oxocycloalkanecarboxylic acid ester.

In the catalytic reaction of the present production method, it is necessary to use an enzyme (the present coenzyme regenerating enzyme) having such an ability that converts again an oxidized-form coenzyme (electron acceptor) that has been produced as a result of consumption of a stoichiometric amount of a reduced-form coenzyme (electron donor) in the asymmetric reducing reaction of 2-oxocycloalkanecarboxylic acid ester into the reduced-form coenzyme (electron donor), in other words, such an ability that regenerates a coenzyme on which the enzyme having an ability to asymmetrically reduce 2-oxocycloalkanecarboxylic acid ester to produce 2-hydroxycycloalkanecarboxylic acid ester depends. In such a case, the present coenzyme regenerating enzyme may be an enzyme which is different from the present reductase which performs the aforementioned asymmetric reducing reaction, or the present reductase may also have the function as a coenzyme regenerating enzyme. Of course, combination of both of the above cases is possible.

In this context, the fact that “the present reductase also has the function as a coenzyme regenerating enzyme” can be confirmed, for example, by determining if a reduced-form coenzyme (electron donor) is produced when a reaction for oxidizing a regeneration system material compound which is a substrate of the coenzyme regenerating enzyme is conducted in the presence of the oxidized-form coenzyme (electron acceptor) using the isolated present reductase.

Examples of the present coenzyme regenerating enzyme include, for example, glucose dehydrogenase, alcohol dehydrogenase, aldehyde dehydrogenase, amino acid dehydrogenase and organic dehydrogenase (such as malic acid dehydrogenase). Among these, coenzyme regenerating enzymes which produce the coenzyme by oxidizing an aliphatic alcohol (for example, alcohols having a boiling point of not more than 200° C. such as 2-propanol, 2-buthanol, 2-penthanol, 2-hyxanol, 2-heptanol and 2-octanol) are preferred. The amount of aliphatic alcohol used herein is not more than 100 times by mole, preferably not more than 10 times by mole with respect to 2-oxocycloalkanecarboxylic acid ester.

The reaction may be conducted, for example, by mixing water, 2-oxocycloalkanecarboxylic acid ester, the present transformant or dead cells thereof, as well as a coenzyme and an organic solvent, if necessary, and stirring and shaking the resultant mixture.

The progress of the reaction can be determined, for example, by following the remaining amount of the material compound in the reaction mixture by means of the liquid chromatography, gas chromatography and the like. The range of the reaction time is usually 5 minutes to 10 days, preferably 30 minutes to 4 days.

After completion of the reaction, the objective substance may be collected by conventionally used method for isolating a compound using an enzyme as a catalyst. For instance, first the reaction mixture is extracted with an organic solvent such as hexane, heptane, tert-butylmethylether, ethyl acetate or toluene. If necessary, filtration of reaction mixture or a treatment such as centrifugal separation for removing impurities may be performed prior to the above extracting operation. Next, the extracted organic phase is dried to thereby obtain the objective substance in the form of a concentrate. The objective substance may be further purified by column chromatography or the like as necessary.

### EXAMPLES

In the following, the present invention will be explained more specifically by way of production examples and the like, however, the present invention is not limited to these examples.

### Example 1

(Preparation of Present Reductase Gene (Part 1) and Preparation of Transformant Containing Present Reductase Gene)

A plasmid pKAR containing a DNA comprising the nucleotide sequence represented by SEQ ID NO: 2 was prepared in the following manner.

First, a DNA fragment including a DNA represented by SEQ ID NO: 2 was cut out from a known plasmid pUAR (Deposit number: FERM BP-7752, which was deposited under the Budapest Treaty on Sep. 21, 2001, transferred from FERM P-18127 deposited on Nov. 27, 2000) as described in, for example, Appl Microbial Biotechnol (1999) 52:386-392 using PstI and SmaI. The cut out DNA fragment was inserted downstream the Tac promoter of the vector pKK223-3 (available from Amersham Pharmacia Biotech Corporation) that had been treated with PstI/SmaI. In this manner, the plasmid pKAR was constructed.

Using the constructed plasmid pKAR, E. coli strain JM109 was transformed.

Next, a flask was charged with 100 mL of liquid culture medium (10 g of triptone, 5 g of yeast extract and 5 g of sodium chloride were dissolved in 1000 mL of water. To this solution 1 N sodium hydroxide aqueous solution was added dropwise to adjust the pH at 7.0), and after sterilization, ampicillin, ZnCl₂ and isopropylthio-β-D-galactoside (IPTG) were added so that the final concentration thereof were 100 μg/mL, 0.01% (w/v) and 0.4 mM, respectively. The culture medium thus prepared was inoculated with 0.3 mL of a culture solution in which the transformant (E.coli strain JM109/pKAR) obtained above had been cultivated in the liquid culture medium having the aforementioned composition, and the culture was incubated under shaking for 14 hours at 30° C.

After incubation, the obtained culture solution was subjected to centrifugal separation (15000×g, 15 min., 4° C.), whereby the bacterial cells were collected. The collected bacterial cells were then suspended in 30 mL of 50 mM potassium dihydrogenphosphate/dipotassium hydrogenphosphate buffer (pH 7.0) and by subjecting the suspension to centrifugal separation (15000×g, 15 min., 4° C.), washed bacterial cells which are transformants containing the present reductase gene were obtained.

### Example 2

(Preparation of Present Reductase Gene (Part 2))

(2-1) Preparation of Chromosomal DNA

A flask is charged with 100 mL of liquid culture medium (5 g of triptone, 2.5 g of yeast extract, 4 g of sodium chloride, 2.5 g of gelatin, 1.5 g of sodium acetate and 2.4 g of threonine are dissolved in 1000 mL of water. To this solution, 1 N sodium hydroxide aqueous solution is added dropwise to adjust the pH at 7.0), and sterilized. The culture medium thus prepared is inoculated with 0.3 mL of a culture solution in which known Corynebacterium pseudodiphteriticum strain ST-10 (Deposit number: FERM P-13150) as disclosed in Japanese Unexamined Patent Publication JP-A 10-94399 etc. has been cultivated in the liquid culture medium having the aforementioned composition, and the culture is incubated under shaking for 10 hours at 30° C.

After incubation, the obtained culture is subjected to centrifugal separation (15000×g, 15 min., 4° C.), whereby the bacterial cells are collected. The collected bacterial cells are then suspended in 30 mL of 50 mM potassium dihydrogenphosphate/dipotassium hydrogenphosphate buffer (pH 7.0) and by subjecting the suspension to centrifugal separation (1500033 g, 15 min., 4° C.), washed bacterial cells are obtained. Using the washed bacterial cells thus obtained, a chromosomal DNA is prepared in accordance with the method of J. C. Wang et al. (Appl Microbiol Biotechnol (1999) 52:386-392).

(2-2) Preparation of Plasmid Containing Present Reductase Gene (Construction of Plasmid pTrcPAR)

A PCR reaction is performed using an oligonucleotide having the nucleotide sequence represented by SEQ ID NO: 5 and an oligonucleotide having the nucleotide sequence represented by SEQ ID NO: 6 as primers and the chromosomal DNA prepared in the above (2-1) as a template in the reaction mixture composition and in the reaction condition as follows (using the “Expand High Fidelity PCR System” available from Roche Diagnostic Corporation).

[Composition of Reaction Mixture]

| | | |
|---|---|---|
| chromosomal DNA | 1 | μl |
| dNTP (each 2.5 mM-mix) | 0.4 | μL |
| primers (20 pmol/μL) | each 0.75 | μL |
| 10 × buffer (with MgCl₂) | 5 | μL |
| enz.expand HiFi (3.5 × 10³ U/mL) | 0.375 μL | |
| ultra pure water | 41.725 | μL |
| [Reaction Condition] | | |

A vessel charged with the reaction mixture having the above composition is placed in the PERKIN ELMER-GeneAmp PCR System 2400, and after heating to 97° C. (2 min.), a cycle of 97° C. (0.25 min.), 55° C. (0.5min.) and 72° C. (1.5 min.) are repeated 10 times, and then a cycle of 97° C. (0.25 min.), 55° C. (0.5 min.) and 72° C. (2.5 min.) is repeated 20 times, followed by 7 min. retention at 72° C.

By adding two kinds restriction enzymes (NcoI and BamHI) to the PCR amplified DNA fragment obtained by purifying the PCR reaction mixture, the DNA fragment is double-digested. Then, the obtained DNA fragment is purified.

On the other hand, by adding two kinds of restriction enzymes (NcoI and BamHI), the vector pTrc99A (available from Pharmacia) is double-digested. Then, the obtained DNA fragment is purified.

Two kinds of DNA fragments thus obtained by purification are mixed, and ligated with T4 DNA ligase. Then, E.coli DH5α is transformed with the ligation solution thus obtained.

A Plasmid containing the present reductase gene (hereinafter, also referred to as plasmid pTrcPAR) is extracted from the obtained transformants using QIAprep Spin Miniprep Kit (available from Qiagen Corporation).

### Example 3

(Preparation of Present Reductase Gene (Part 3))

(3- 1) Preparation of cDNA Library

A 500 mL flask was charged with 100 mL of culture medium (potato dextrose broth (available from Becton Dickinson Corporation) was dissolved in water in the concentration of 24 g/L) and sterilized for 15 minutes at 121° C. To this solution was added 0.5 mL of culture solution of Penicillium citrinum strain IF04631 having cultivated in the culture medium of the same composition (30° C., 48 hours, shaking cultivation) and incubated for 72 hours at 30° C. under shaking. Thereafter, the obtained culture solution was subjected to centrifugal separation (8000×g, 10 min.) to collect the produced precipitate. This precipitate was washed with 50 mL of 20 mM potassium phosphate buffer (pH 7.0) three times, and about 1.0 g of washed bacterial cells were obtained.

Using the washed bacterial cells of Penicillium citrinum strain IF04631, a whole RNA was prepared in accordance with guanidine thiocyanate phenol chloroform method. From the whole RNA thus prepared, an RNA having poly(A) was obtained using Oligotex(dT)30-Super (available from TAKARA SYUZO CO., LTD.).

Preparation of the cDNA library was performed in accordance with the Gubler and Hoffman method. First, using the RNA having poly(A) as obtained above, Oligo(dT)18-linker-primer ((XhoI containing site) available from TAKARA SYUZO CO., LTD.), RAV-2 Rtase and SuperScriptII Rtase, a single strand cDNA was prepared. To the prepared single strand cDNA (in the form of solution containing the cDNA) was added E. coli DNA polymerase, E.coliRnase/E.coli DNA Ligase Mixture and T4 DNA Polymerase to perform synthesis of a double strand cDNA and blunt-end manipulation for the double strand cDNA.

Ligation between the double strand cDNA thus obtained and an EcoRI-NotI-BamHI adopter (available from TAKARA SYUZO CO., LTD.) was then conducted. The DNA obtained after ligation was subjected to phosphorylation, fragmentation with XhoI, removal of low molecular weight DNAs using spin column (available from TAKARA SYUZO CO., LTD.) and ligation with λ ZapII (EcoRI-XhoI fragment), followed by packaging with the use of in vitro packaging kit (available from STRATAGENE Corporation) to thereby prepare a cDNA library (hereinafter, also referred to as cDNA library (A)).

(3-2) Preparation of Plasmid Containing Present Reductase Gene (Construction of Plasmid pTrcRPc)

A PCR reaction was performed using an oligonucleotide having the nucleotide sequence represented by SEQ ID NO: 7 and an oligonucleotide having the nucleotide sequence represented by SEQ ID NO: 8 as primers and the cDNA library prepared in the above (3-1) as a template in the reaction mixture composition and in the reaction condition as follows (using the “Expand High Fidelity PCR System” available from Roche Diagnostic Corporation).

[Composition of Reaction Mixture]

| | | |
|---|---|---|
| eDNA library cone. solution | 1 | μL |
| dNTP (each 2.5 mM-mix) | 0.4 | μL |
| primers (20 pmol/μL) | each 0.75 | μL |
| 10 × buffer (with MgCl₂) | 5 | μL |
| enz.expand HiFi (3.5 × 10³ U/mL) | 0.375 | μL |
| ultra pure water | 41.725 | μL |
| [Reaction Condition] | | |

A vessel charged with the reaction mixture having the above composition was placed in the PERKIN ELMER-GeneAmp PCR System 2400, and after heating to 97° C. (2 min.), a cycle of 97° C. (0.25 min.), 55° C. (0.5 min.) and 72° C. (1.5 min.) were repeated 10 times, and then a cycle of 97° C. (0.25 min.), 55° C. (0.5 min.) and 72° C. (2.5 min.) was repeated 20 times, followed by 7 min. retention at 72° C.

By adding two kinds restriction enzymes (NcoI and BamHI) to the PCR amplified DNA fragment obtained by purifying the PCR reaction mixture, the DNA fragment was double-digested. Then the obtained DNA fragment was purified.

On the other hand, by adding two kinds of restriction enzymes (NcoI and BamHI), the vector pTrc99A (available from Pharmacia) was double-digested. Then the obtained DNA fragment was purified.

Two kinds of DNA fragments thus obtained by purification were mixed, and ligated with T4 DNA ligase. Then, E.coli DH5α was transformed with the ligation solution thus obtained.

A Plasmid containing the present reductase gene (hereinafter, also referred to as plasmid pTrcRPc) was extracted from the obtained transformants using QIAprep Spin Miniprep Kit (available from Qiagen Corporation).

### Example 4

(Preparation of Present Coenzyme Regenerating Enzyme Gene)

(4-1) Arrangement for Preparing Gene Having Nucleotide Sequence Encoding Amino Acid Sequence of Enzyme Which has Ability to Convert Oxidized-Form β-nicotineamide Adenine Dinucleotide or the Like Into its Reduced Form.

A flask was charged with 100 mL of LB medium (1% triptone, 0.5% yeast extract and 1% sodium chloride) and sterilized. The culture medium thus prepared was inoculated with 0.3 mL of culture solution in which Bacillus megaterium strain IF012108 had been cultivated in the liquid culture medium having the aforementioned composition, and was incubated for 10 hours at 30° C. under shaking.

After incubation, the obtained culture was subjected to centrifugal separation (15000×g, 15 min., 4° C.) to collect the bacterial cells. The collected bacterial cells were then suspended in 30 mL of 50 mM potassium dihydrogenphosphate/dipotassium hydrogenphosphate buffer (pH 7.0) and by subjecting the suspension to centrifugal separation (15000×g, 15 min., 4° C.), washed bacterial cells were obtained. From the washed bacterial cells thus obtained, a chromosomal DNA was purified using Qiagen Genomic Tip (available from Qiagen Corporation) in accordance with the manual attached thereto.

(4-2) Preparation of Gene Having Nucleotide Sequence Encoding Amino Acid Sequence of Enzyme Which Has Ability to Convert Oxidized-Form β-nicotineamide Adenine Dinucleotide or the Like Into Its Reduced Form (Part 1: Construction of Plasmid pSDGDH12)

An oligonucleotide having the nucleotide sequence represented by SEQ ID NO: 9 and an oligonucleotide having the nucleotide sequence represented by SEQ ID NO: 10 were synthesized on the basis of the amino acid sequence of the known glucose dehydrogenase derived from Bacillus megaterium IWG3 as described in The Journal of Biological Chemistry Vol.264, No.11, 6381-6385 (1989).

A PCR was conducted while using the oligonucleotide having the nucleotide sequence represented by SEQ ID NO: 9 and the oligonucleotide having the nucleotide sequence represented by SEQ ID NO: 10 as primers and the chromosomal DNA purified in the above (4-1) as a template, in the same composition of reaction mixture and in the same reaction condition as described in (2-2) of Example 2 (using Expand-High Fidelity PCR System available from Roche Diagnostic Corporation).

The PCR-amplified DNA fragment obtained by purification of the PCR reaction mixture was ligated into the existing “PCR Product insertion site” of the vector pCR2.1-TOPO using TOPO™TA cloning kit available from Invitrogen Corporation. Then, E.coli DH5α was transformed with the ligation solution thus obtained.

From the transformant thus obtained, a plasmid containing glucose dehydrogenase (hereinafter, also referred to as plasmid pSDGDH12) was extracted by using QIAprep Spin Miniprep Kit (available from Qiagen Corporation).

Next, a sequencing reaction was carried out using the extracted plasmid pSDGDH12 as a template by means of Dye Terminator cycle sequencing FS ready Reaction Kit (available from Perkin-Elmer Corporation), and the nucleotide sequence of obtained DNA was analyzed using DNA sequencer 373A (available from Perkin-Elmer Corporation). The result is shown as SEQ ID NO: 11.

(4-3) Preparation of Gene Having Nucleotide Sequence Encoding Amino Acid Sequence of Enzyme Which Has Ability to Convert Oxidized-Form β-Nicotineamide Adenine Dinucleotide Phosphate or the Like Into Its Reduced Form (Part 2: Construction of Plasmid pAGDH12)

(4-3-1) Construction of Plasmid pTGDH12

An oligonucleotide having the nucleotide sequence represented by SEQ ID NO: 13 and an oligonucleotide having the nucleotide sequence represented by SEQ ID NO: 14 were synthesized on the basis of the nucleotide sequence represented by SEQ ID NO: 11.

A PCR was conducted while using the oligonucleotide having the nucleotide sequence represented by SEQ ID NO: 13 and the oligonucleotide having the nucleotide sequence represented by SEQ ID NO: 14 as primers and the chromosomal DNA purified in the above (4-1) as a template, in the reaction mixture condition and the reaction condition as follows (using Expand-High Fidelity PCR System available from Roche Diagnostic Corporation).

[Composition of Reaction Mixture]

| | | |
|---|---|---|
| chromosomal DNA conc. solution | 1 | μL |
| dNTP (each 2.5 mM-mix) | 0.4 | μL |
| primers (20 pmol/μL) | each 0.75 | μL |
| 10 x buffer (with MgCl2) | 5 | μL |
| enz.expand HiFi (3.5 × 10³ U/mL) | 0.375 | μL |
| ultra pure water | 41.725 | μL |
| [PCR Reaction Condition] | | |

A vessel charged with the reaction mixture having the above composition was placed in the PERKIN ELMER-GeneAmp PCR System 2400, and after heating to 97° C. (2 min.), a cycle of 97° C. (0.25 min.), 55° C. (0.5 min.) and 72° C. (1.5 min.) were repeated 10 times, and then a cycle of 97° C. (0.25 min.), 55° C. (0.5 min.) and 72° C. (2.5 min.) was repeated 20 times, followed by 7 min. retention at 72° C.

By adding two kinds restriction enzymes (NcoI and BamHI) to the PCR amplified DNA fragment obtained by purifying the PCR reaction mixture, the DNA fragment was double-digested. Next, the obtained DNA fragment was purified.

On the other hand, by adding two kinds of restriction enzymes (NcoI and BamHI), the vector pTV118N (available from TAKARA SYUZO Co., Ltd.) was double-digested. Next, the obtained DNA fragment was purified.

Two kinds of DNA fragments thus obtained by purification were mixed, and ligated with T4 DNA ligase. Then, E.coli DH5α was transformed with the ligation solution thus obtained. A plasmid containing the present reductase gene (hereinafter, also referred to as plasmid pTGDH12) was extracted from the obtained transformant using QIAprep Spin Miniprep Kit (available from Qiagen Corporation).

(4-3-2) Construction of Plasmid pCGDH12

An oligonucleotide having the nucleotide sequence represented by SEQ ID NO: 15 was synthesized on the basis of the nucleotide sequence of vector pTV118N (available from TAKARA SYUZO Co., Ltd.).

A PCR was conducted while using the oligonucleotide having the nucleotide sequence represented by SEQ ID NO: 15 and the oligonucleotide having the nucleotide sequence represented by SEQ ID NO: 14 as primers and the plasmid pTGDH12 as a template, in the reaction mixture condition and the reaction condition as follows (using Expand-High Fidelity PCR System available from Roche Diagnostic Corporation).

[Composition of Reaction Mixture]

| | | |
|---|---|---|
| plasmid pTGDH12 solution | 1 | L |
| dNTP (each 2.5 mM-mix) | 0.4 | μL |
| primers (20 pmol/j.tL) | each 0.75 | μL |
| 10 × buffer (with MgCl₂) | 5 | μL |
| enz.expand HiFi (3.5 × 10³ U/mL) | 0.375 | μL |
| ultra pure water | 41.725 | μL |
| [PCR Reaction Condition] | | |

A vessel charged with the reaction mixture having the above composition was placed in the PERKIN ELMER-GeneAmp PCR System 2400, and after heating to 97° C. (2 min.), a cycle of 97° C. (0.25 min.), 55° C. (0.5 min.) and 72° C. (1.5 min.) were repeated 10 times, and then a cycle of 97° C. (0.25 min.), 55° C. (0.5 min.) and 72° C. (2.5 min.) was repeated 20 times, followed by 7 min. retention at 72° C.

Using the PCR reaction solution thus obtained and TOPO™TA cloning kit Ver. E available from Invitrogen Corporation, a DNA fragment of about 1000 bp obtained by the PCR was ligated into the existing “PCR Product insertion site”of the vector pCR2.1-TOPO, and E.coli DH5α was transformed with the ligation solution thus obtained. From the transformant thus obtained, a plasmid containing the present reductase gene (hereinafter, also referred to as plasmid pCGDH12) was extracted by using QIAprep Spin Miniprep Kit (available from Qiagen Corporation).

(4-3-3) Construction of Plasmid pAGDH12

By adding a restriction enzyme (BamHI) to plasmid pCGDH12, the plasmid was digested. Then the obtained DNA fragment (about 1000 bp) was purified.

On the other hand, by adding a restriction enzyme (BamHI) to vector pACYC184 (available from NIPPON GENE Corporation), the vector, was digested. Then the obtained DNA fragment was purified. Additionally, for preventing self ligation, dephosphorylation was conducted by using Alkaline Phospatase (available from TAKARA SYUZO CO., LTD.).

Two kinds of DNA fragments thus obtained by conducting purification in the above manner were mixed, and ligated using T4 DNA ligase. Then E.coli DH5α was transformed with the ligation solution thus obtained. The obtained transformant was cultivated on the LB agar medium containing 20 μg/mL of chloramphenicol, and 4 colonies were selected at random from grown-up colonies. A sterilized LB medium (2 mL) containing 20 μg/mL of chloramphenicol was inoculated with each of these selected colonies, and incubated under shaking in a test tube (30° C., 24 hours). Plasmids were extracted from each bacterial culture, by using QIA prep Spin Miniprep Kit (available from Qiagen Corporation). The respective plasmids thus extracted were partly digested by a restriction enzyme (BamHI), and the digest was applied to the electrophoresis to confirm that all the extracted plasmids included said DNA fragment (about 1000 bp) inserted therein (hereinafter, the extracted plasmid is also referred to as plasmid pAGDH12).

### Example 5

(Preparation of Plasmid Containing Present Reductase Gene and Present Coenzyme Regenerating Enzyme Gene (Part 1): Construction of Plasmid pTrcPARSbG)

By adding two kinds of restriction enzymes (BamHI and XbaI) to the plasmid pSDGDH12 prepared in (4-2) of Example 4, the plasmid is double-digested. Then the digested DNA fragment is purified.

On the other hand, by adding two kinds of restriction enzymes (BamHI and XbaI) to the plasmid pTrcPAR prepared in Example 2, the plasmid is double-digested. Then the digested DNA fragment is purified.

Two kinds of DNA fragments thus obtained by conducting purification in the above manner are mixed, and ligated using T4 DNA ligase. E.coli DH5α is transformed with the ligation solution thus obtained. From the transformant, a plasmid containing the present reductase gene and the present coenzyme regenerating enzyme gene (hereinafter also referred to as plasmid pTrcPARSbG) is extracted by using QIA prep Spin Miniprep Kit (available from Qiagen Corporation).

### Example 6

(Preparation of Plasmid Containing Present Reductase Gene and Present Coenzyme Regenerating Enzyme Gene (Part 2): Construction of Plasmid pTrcRSbG12)

By adding two kinds of restriction enzymes (BamHI and XbaI) to the plasmid pSDGDH12 prepared in (4-2) of Example 4, the plasmid was double-digested. Then the digested DNA fragment was purified.

On the other hand, by adding two kinds of restriction enzymes (BamHI and XbaI) to the plasmid pTrcRPc prepared in Example 3, the plasmid was double-digested. Then the digested DNA fragment was purified.

Two kinds of DNA fragments thus obtained by conducting purification in the above manner were mixed, and ligated using T4 DNA ligase. E.coli DH5α was transformed with the ligation solution thus obtained. From the transformant, a plasmid containing the present reductase gene and the present coenzyme regenerating enzyme gene (hereinafter also referred to as plasmid pTrcRSbG12) was extracted by using QIA prep Spin Miniprep Kit (available from Qiagen Corporation).

### Example 7

(Preparation of Transformant Containing Present Reductase Gene and Present Coenzyme Regenerating Enzyme Gene (Part 1))

E.coli HB101 is transformed with the plasmid pTrcPARSbG prepared in Example 5. A set (100 mL×3) of sterilized LB medium including 0.4 mM IPTG, 0.01% (w/v) ZnCl₂ and 50 μg/mL ampicillin is inoculated with the obtained transformant, and incubated under shaking (30° C., 18 hours). After incubation, the culture solution is subjected to centrifugal separation and washing, to collect washed bacterial cells.

### Example 8

(Preparation of Transformant Containing Present Reductase Gene and Present Coenzyme Regenerating Enzyme Gene (Part 2))

E.coli HB101 was transformed with the plasmid pTrcRSbG12 prepared in Example 6. A set (100 mL×3) of sterilized LB medium including 0.1 mM IPTG, and 50 μg/mL ampicillin was inoculated with the obtained transformant and incubated under shaking (30° C., 18 hours). After incubation, the culture was subjected to centrifugal separation and washing, to collect 1.2 g of washed bacterial cells.

### Example 9

(Preparation of Transformant Containing Present Reductase Gene and Present Coenzyme Regenerating Enzyme Gene (Part 3))

E.coli HB101 was transformed with the plasmid pTrcRPc prepared in Example 3 and with the plasmid pAGDH12 prepared in (4-3) of Example 4. A set (100 mL×3) of sterilized LB medium including 0.4 mM IPTG, 20 μg/mL chloramphenicol and 50 μg/mL ampicillin was inoculated with the obtained transformant and incubated under shaking (30° C., 18 hours). After incubation, the culture solution was subjected to centrifugal separation and washing, to collect washed bacterial cells.

### Reference Example 1

(Production of Optically Active 2-hydroxycycloalkanecarboxylic Acid Ester)

To 25 mL of 100 mM potassium dihydrogenphosphate/dipotassium hydrogenphosphate buffer (pH 7.0), 2 g of washed bacterial cells prepared in Example 1, 13.3 mg of NAD⁺ and 5%(v/v) 2-propanol were added. To this mixture was further added 96 mg of ethyl 2-oxocyclohexane carboxylate. The mixture (reaction mixture) thus obtained was stirred for 19 hours at 30° C., thereby allowing the reaction. After completion of the reaction, 50 mL of ethyl acetate was poured to the reaction mixture and stirred, followed by centrifugal separation for separately collecting the organic phase and the aqueous phase. To the collected aqueous phase, 25 mL of ethyl acetate was added again, and the same manipulation was repeated. After concentrating the combined organic phase, the concentrate was dissolved in 30 mL of chloroform and dried with anhydrous Na₂SO₄. After drying, chloroform was distilled off to obtain 100 mg of optically active ethyl 2-hydroxycyclohexanecarboxylate.

Results of instrumental analysis for the obtained ethyl 2-hydroxycyclohexanecarboxylate were as follows.

<Results of Instrumental Analysis>

Chemical purity 79%(gas chromatogram peak area ratio of the compound), cis/trans=95/5(gas chromatogram peak area ratio of the compound), specific rotation power [α] D=19° (chloroform, 25° C., C=0.7), absolute configuration of product (1R,2S)

The absolute configuration of the obtained ethyl 2-hydroxycyclohexanecarboxylate was determined by comparison of retention time with a cis isomer and a trans isomer of commercially available ethyl 2-hydroxycyclohexanecarboxylate in accordance with the gas chromatography (GC) analysis under the analysis condition as shown below. The absolute configuration was determined while referring to the data disclosed in a reference paper (Chem. Lett. (1989) 1465-1466), [specific rotatory power of ethyl (1R,2S)-2-hydroxycyclohexanecarboxylate [α] D=25.87′ (chloroform, 25° C., C=1.23)].

<Condition of Chemical Purity Analysis>

Gas chromatography

Column: DB-1(J&W Scientific) 0.53 mmφ×30 m 1.5 μm

Inlet temperature: 120° C.

Column room temperature: 70° C. (4° C./min.) to 170° C.

Detection temperature: 300° C.

Carrier gas: helium 10 mL/min.

ethyl cis-2-hydroxycyclohexanecarboxylate 15.6 min.

ethyl trans-2-hydroxycyclohexanecarboxylate 15.9 min.

### Example 10

(Method for Producing 2-hydroxycycloalkanecarboxylic Acid Ester (Part 1))

To 20 mL of 100 mM potassium dihydrogenphosphate/dipotassium hydrogenphosphate buffer (pH 6.5), 1 g of washed bacterial cells prepared in Example 7, 12 mg of NAD⁺ and 2.5 g of glucose are added. To this mixture is further added 240 mg of ethyl 2-oxocyclohexane carboxylate, and then the pH of the resultant mixture is adjusted at 6.5 with 15% sodium carbonate aqueous solution. The mixture (reaction mixture) thus obtained is stirred for 4 hours at 30° C. for allowing the reaction. After completion of the reaction, 25 mL of ethyl acetate is poured to the reaction mixture and stirred, followed by centrifugal separation for separately collecting the organic phase and the aqueous phase. To the collected aqueous phase, 25 mL of ethyl acetate is added again, and the same manipulation is repeated. After concentrating the organic phase, the concentrate is dissolved in 30 mL of chloroform and dried with anhydrous Na₂SO₄. After drying, chloroform is distilled off to obtain ethyl 2-hydroxycyclohexane carboxylate.

### Example 11

(Production of Optically Active 2-hydroxycycloalkanecarboxylic Acid Ester (Part 2))

To 20 mL of 100 mM potassium dihydrogenphosphate/dipotassium hydrogenphosphate buffer (pH 6.5), 1 g of washed bacterial cells prepared in Example 8, 12 mg of NADP⁺ and 2.5 g of glucose were added. To this mixture was further added 240 mg of ethyl 2-oxocyclohexane carboxylate, and then the pH of the resultant mixture was adjusted at 6.5 with 15% sodium carbonate aqueous solution. The mixture (reaction mixture) thus obtained was stirred for 4 hours at 30° C. for allowing the reaction. After completion of the reaction, 25 mL of ethyl acetate was poured to the reaction mixture and stirred, followed by centrifugal separation for separately collecting the organic phase and the aqueous phase. To the collected aqueous phase, 25 mL of ethyl acetate was added again, and the same manipulation was repeated. After concentrating the combined organic phase, the concentrate was dissolved in 30 mL of chloroform and dried with anhydrous Na₂SO₄. After drying, chloroform was distilled off to obtain 220 mg of optically active ethyl 2-hydroxycyclohexanecarboxylate.

Results of instrumental analysis of the obtained ethyl 2-hydroxycyclohexanecarboxylate were as follows.

<Results of Instrumental Analysis>

Chemical purity 99% (gas chromatogram peak area ratio of the compound), cis/trans=99.5/0.5 (gas chromatogram peak area ratio), specific rotatory power [α] D=24° (chloroform, 25° C., C=1), absolute configuration of product (1R,2S)

The absolute configuration of the obtained ethyl 2-hydroxycyclohexanecarboxylate was determined by comparison of retention time with a cis isomer and a trans isomer of commercially available ethyl 2-hydroxycyclohexanecarboxylate in accordance with the gas chromatography (GC) analysis under the analysis condition as shown below. The absolute configuration was determined while referring to the data disclosed in a reference (Chem. Lett. (1989) 1465-1466), [specific rotatory power of ethyl (1R,2S)-2-hydroxycyclohexanecarboxylate [α] D=25.87°(chloroform, 25° C., C=1.23)].

<Condition of Chemical Purity Analysis>

Gas chromatography

Column: DB-1(J&W Scientific) 0.53 mmφ×30 m 1.5 μm

Inlet temperature: 120° C.

Column room temperature: 70° C. (4° C./min.) to 170° C.

Detection temperature: 300° C.

Carrier gas: helium 10 mL/min.

ethyl cis-2-hydroxycyclohexanecarboxylate 15.6 min.

ethyl trans-2-hydroxycyclohexanecarboxylate 15.9 min.

### Example 12

(Production of an Optically Active 2-hydroxycycloalkanecarboxylic Acid Ester (Part 3))

To 20 mL of 100 mM potassium dihydrogenphosphate/dipotassium hydrogenphosphate buffer (pH 6.5), 1 g of washed bacterial cells prepared in Example 9, 12 mg of NADP⁺ and 2.5 g of glucose are added. To this mixture is further added 240 mg of ethyl 2-oxocyclohexane carboxylate, and then the pH of the resultant mixture is adjusted at 6.5 with 15% sodium carbonate aqueous solution. The mixture (reaction mixture) thus obtained is stirred for 4 hours at 30° C. for allowing the reaction. After completion of the reaction, 25 mL of ethyl acetate is poured to the reaction mixture and stirred, followed by centrifugal separation for separately collecting the organic phase and the aqueous phase. To the collected aqueous phase, 25 mL of ethyl acetate is added again, and the same manipulation is repeated. After concentrating the organic phase, the concentrate is dissolved in 30 mL of chloroform and dried with anhydrous Na₂SO₄. After drying, chloroform is distilled off to obtain ethyl 2-hydroxycyclohexane carboxylate.

### Reference Example 2

(2-1) Obtaining Microorganisms That Produce Optically Active 2-hydroxycycloalkanecarboxylic Acid Ester

After inoculating a sterilized LB medium (10 mL) with commercially-available microorganisms or microorganism isolated from soil or the like, the culture medium is incubated under shaking (30° C., 18 hours). After the incubation, the culture solution is subjected to centrifugal separation and washing to collect washed bacterial cells.

(2-2) Screening

To 20 mL of 100 mM potassium dihydrogenphosphate/dipotassium hydrogenphosphate buffer (pH 6.5), 1 g of washed bacterial cells prepared in the above (2-1), 12 mg of NADP⁺, 12 mg of NAD⁺ and 2.5 g of glucose are added. To this mixture is further added 240 mg of ethyl 2-oxocyclohexane carboxylate, and then the pH of the resultant mixture is adjusted at 6.5 with 15% sodium carbonate aqueous solution. The mixture (reaction mixture) thus obtained is stirred for 4 hours at 30° C. for allowing the reaction. After completion of the reaction, 25 mL of ethyl acetate is poured to the reaction mixture and stirred, followed by centrifugal separation for separately collecting the organic phase and the aqueous phase. To the collected aqueous phase, 25 mL of ethyl acetate is added again, and the similar operation is repeated. After concentrating the organic phase, the concentrate is dissolved in 30 mL of chloroform and dried with anhydrous Na₂SO₄. After drying, chloroform is distilled off to obtain a residue. Presence of ethyl 2-hydroxycyclohexanecarboxylate in the obtained residue is confirmed by a qualitative and/or quantitative analysis by way of liquid chromatography or gas chromatography (also optical purity analysis is possible). In connection with this, since the absolute configuration of ethyl 2-hydroxycyclohexanecarboxylate can be determined by referring the data of the reference (Tetrahedron Lett. (1986) 2631-2634), [specific rotatory power of ethyl (1S, 2S)-2- hydroxycyclohexanecarboxylate [α] D=+58° (diethylether, 20° C., C=0.5)], microorganisms that produce ethyl (1S,2S)-2-hydroxycyclohexanecarboxylate can also be selected. The following is a description of the analysis condition in the gas chromatography.

<Condition of Chemical Purity Analysis>

Gas chromatography

Column: DB-1(J&W Scientific) 0.53 mmφ×30 m 1.5 μm

Inlet temperature: 120° C.

Column room temperature: 70° C. (4° C./min.) to 170° C.

Detection temperature: 300° C.

Carrier gas: helium 10 mL/min.

ethyl cis-2-hydroxycyclohexanecarboxylate: 15.6 min.

ethyl trans-2-hydroxycyclohexanecarboxylate: 15.9 min.

Effect of the Invention

According to the present invention, optically active 2-hydroxycycloalkanecarboxylic acid ester, which is a useful intermediate for the production of bioactive substance can be readily obtained.

[Sequence List Free Text]

SEQ ID NO: 5

Oligonucleotide which is a primer designed for PCR

SEQ ID NO: 6

Oligonucleotide which is a primer designed for PCR

SEQ ID NO: 7

Oligonucleotide which is a primer designed for PCR

SEQ ID NO: 8

Oligonucleotide which is a primer designed for PCR

SEQ ID NO: 9

Oligonucleotide which is a primer designed for PCR

SEQ ID NO: 10

Oligonucleotide which is a primer designed for PCR

SEQ ID NO: 13

Oligonucleotide which is a primer designed for PCR

SEQ ID NO: 14

Oligonucleotide which is a primer designed for PCR

SEQ ID NO: 15

Oligonucleotide which is a primer designed for PCR

What is claimed is:

1. A method for producing optically active 2-hydroxycycloalkanecarboxylic acid ester comprising the steps of:
(a) allowing 2-oxocycloalkanecarboxylic acid ester to react with a transformant or a dead cell thereof artificially provided with
(i) an ability to asymmetrically reduce 2-oxocycloalkanecarboxylic acid ester to optically active 2-hydroxycycloalkanecarboxylic acid ester, and
(ii) an ability to regenerate a coenzyme on which an enzyme having the ability as defined in (i) depends; and
(b) collecting the resulting optically active 2-hydroxycycloalkanecarboxylic acid ester.

2. The production method according to claim 1, wherein the transformant is a transformant having at least one selected from:
(A) a plasmid comprising a nucleotide sequence encoding an amino acid sequence of an enzyme having both of the two abilities (i) and (ii) as described below;
(B) a plasmid comprising
(a) a DNA having a nucleotide sequence encoding an amino acid sequence of an enzyme having an ability (i) as described below, and
(b) a DNA having a nucleotide sequence encoding an amino acid sequence of an enzyme having an ability (ii) as described below; or
(C) a pair of plasmids:
(a) a plasmid comprising a DNA having a nucleotide sequence encoding an amino acid sequence of an enzyme having an ability (i) as described below, and
(b) a plasmid comprising a DNA having a nucleotide sequence encoding an amino acid sequence of an enzyme having an ability (ii) as described below,
wherein said abilities are:
(i) an ability to asymmetrically reduce 2-oxocycloalkanecarboxylic acid ester to optically active 2-hydroxycycloalkanecarboxylic acid ester; and
(ii) an ability to regenerate a coenzyme on which an enzyme having the above ability (i) depends.

3. The production method according to claim 1, wherein the transformant is Escherichia coli.

4. The production method according to calim 1, wherein the coenzyme is NADH/NAD⁺ (nicotinamide adenine dinucleotide) or NADPH/NADP⁺ (nicotinamide adenine dinucleotide phosphate).

5. The production method according to claim 1, wherein the 2-oxocycloalkane carboxylic acid ester is allowed to react with the transformant or a dead cell thereof in the presence of an aliphatic alcohol.

6. The production method according to claim 5, whrein the aliphatic alcohol is an alcohol having a boiling point of not more than 200° C.

7. The production method according to claim 5, wherein the aliphatic alcohol is 2-propanol.

8. The production method according to claim 1, wherein the 2-oxocycloalkanecarboxylic acid ester is allowed to react with the transformant or a dead cell thereof in the presence of glucose.

9. The production method according to claim 1, wherein the ability to asymmetrically reduce 2-oxocycloalkanecarboxylic acid ester to optically active 2-hydroxycycloalkanecarboxylic acid ester is an ability possessed by an enzyme having an amino acid sequence selected from the group consisting of the following amino acid sequences:
(a) an amino acid sequence represented by SEQ ID NO:1 or 3;
(b) an amino acid sequence represented by SEQ ID NO: 1 or 3 in which one or more amino acids are deleted, substituted or added, and the amino acid sequence is an amino acid sequence of an enzyme having an ability to asymmetrically reduce 2-oxocycloalkanecarboxylic acid ester to optically active 2-hydroxycycloalkanecarboxylic acid ester;
(c) an amino acid sequence encoded by the nucleotide sequence represented by SEQ ID NO: 2 or 4;
(d) an amino acid sequence encoded by a nucleotide sequence of a DNA which hybridizes with a DNA consisting of the nucleotide sequence represented by SEQ ID NO: 2 or 4 under the stringent condition, and the amino acid sequence is an amino acid sequence of an enzyme having an ability to asymmetrically reduce 2-oxocycloalkanecarboxylic acid ester to optically active 2-hydroxycycloalkanecarboxylic acid ester; and
(e) an amino acid sequence of an enzyme,
having an ability to asymmetrically reduce 2-oxocycloalkanecarboxylic acid ester to optically active 2-hydroxycycloalkanecarboxylic acid ester, and
derived from microorganism of genus Corynebacterium or genus Penicillium.

10. The production method according to claim 1, wherein the ability to asymmetrically reduce 2-oxocycloalkanecarboxylic acid ester to optically active 2-hydroxycycloalkanecarboxylic acid ester is an ability possessed by an enzyme having an amino acid sequence selected from the group consisting of the following amino acid sequences:
(a) an amino acid sequence represented by SEQ ID NO: 1;
(b) an amino acid sequence represented by SEQ ID NO: 1 in which one or more amino acids are deleted, substituted or added, and the amino acid sequence is an amino acid sequence of an enzyme having an ability to asymmetrically reduce 2-oxocycloalkanecarboxylic acid ester to optically active 2-hydroxycycloalkanecarboxylic acid ester;
(c) an amino acid sequence encoded by the nucleotide sequence represented by SEQ ID NO: 2;
(d) an amino acid sequence encoded by a nucleotide sequence of a DNA that hybridizes with a DNA consisting of the nucleotide sequence represented by SEQ ID NO: 2 under the stringent condition, and the amino acid sequence is an amino acid sequence of an enzyme having an ability to asymmetrically reduce 2-oxocycloalkanecarboxylic acid ester to optically active 2-hydroxycycloalkanecarboxylic acid ester; and
(e) an amino acid sequence of an enzyme,
having an ability to asymmetrically reduce 2-oxocycloalkanecarboxylic acid ester to optically active 2-hydroxycycloalkanecarboxylic acid ester, and
derived from microorganism of genus Corynebacterium.

11. The production method according to claim 1, wherein the ability to asymmetrically reduce 2-oxocycloalkanecarboxylic acid ester to optically active 2-hydroxycycloalkanecarboxylic acid ester is an ability possessed by an enzyme having an amino acid sequence selected from the group consisting of the following amino acid sequences:
(a) an amino acid sequence represented by SEQ ID NO: 3;
(b) an amino acid sequence represented by SEQ ID NO: 3 in which one or more amino acids are deleted, substituted or added, and the amino acid sequence is an amino acid sequence of an enzyme having an ability to asymmetrically reduce 2-oxocycloalkanecarboxylic acid ester to optically active 2-hydroxycycloalkanecarboxylic acid ester;
(c) an amino acid sequence encoded by the nucleotide sequence represented by SEQ ID NO: 4;
(d) an amino acid sequence encoded by a nucleotide sequence of a DNA that hybridizes with a DNA consisting of the nucleotide sequence represented by SEQ ID NO: 4 under the stringent condition, and the amino acid sequence is an amino acid sequence of an enzyme having an ability to asymmetrically reduce 2-oxocycloalkanecarboxylic acid ester to optically active 2-hydroxycycloalkanecarboxylic acid ester; and
(e) an amino acid sequence of an enzyme,
having an ability to asymmetrically reduce 2-oxocycloalkanecarboxylic acid ester to optically active 2-hydroxycycloalkanecarboxylic acid ester, and
derived from microorganism of genus Penicillium.

12. The production method according to claim 1, wherein the ability to asymmetrically reduce 2-oxocycloalkanecarboxylic acid ester to optically active 2-hydroxycycloalkanecarboxylic acid ester is an ability possessed by an enzyme having an amino acid sequence represented by SEQ ID NO: 1.

13. The production method according to claim 1, wherein the ability to asymmetrically reduce 2-oxocycloalkanecarboxylic acid ester to optically active 2-hydroxycycloalkanecarboxylic acid ester is an ability possessed by an enzyme having an amino acid sequence represented by SEQ ID NO: 3.

14. The production method according to claim 1, wherein the ability to asymmetrically reduce 2-oxocycloalkanecarboxylic acid ester to optically active 2-hydroxycycloalkanecarboxylic acid ester is an ability possessed by an enzyme having an amino acid sequence encoded by the nucleotide sequence represented by SEQ ID NO: 2.

15. The production method according to claim 1, wherein the ability to asymmetrically reduce 2-oxocycloalkanecarboxylic acid ester to optically active 2-hydroxycycloalkanecarboxylic acid ester is an ability possessed by an enzyme having an amino acid sequence encoded by the nucleotide sequence represented by SEQ ID NO: 4.

16. Use of a transformant or a dead cell thereof artificially provided with
an ability to asymmetrically reduce 2-oxocycloalkanecarboxylic acid ester to optically active 2-hydroxycycloalkanecarboxylic acid ester, and
an ability to regenerate a coenzyme on which the enzyme having the former ability depends, as a catalyst, for asymmetrically reducing 2-oxocycloalkanecarboxylic acid ester to optically active 2-hydroxycycloalkanecarboxylic acid ester.


SEQUENCE LISTING (15 sequences)

SEQ ID NO: 1 - protein, 385 aa, Corynebacterium pseudodiphtheriticum
MKAIQYTRIGAEPELTEIPKPEPGPGEVLLEVTAAGVCHSDDFIMSLPEEQYTYGLPLTLGHEGAGKVAAVGEGVEGLDIGTNVVVYGPWGCGNCWHCSQGLENYCSRAQELGINPPGLGAPGALAEFMIVDSPRHLVPIGDLDPVKTVPLTDAGLTPYHAIKRSLPKLRGGSYAVVIGTGGLGHVAIQLLRHLSAATVIALDVSADKLELATKVGAHEVVLSDKDAAENVRKITGSQGAALVLDFVGYQPTIDTAMAVAGVGSDVTIVGIGDGQAHAKVGFFQSPYEASVTVPYWGARNELIELIDLAHAGIFDIGGGDLQSRQRCRSVSTTGCRNAQRPCGCGPWSVVPTAVERQRKNTDARPNSIRPGISVRNSVCASCTPR

SEQ ID NO: 2 - DNA, 1158 bp, Corynebacterium pseudodiphtheriticum
  CDS (1)..(1158)
atgaaggcgatccagtacacgcgaatcggcgcggaacccgaactcacggagattcccaaacccgagcccggtccaggtgaagtgctcctggaagtcaccgctgctggcgtctgccactcggacgacttcatcatgagcctgcccgaagagcagtacacctacggccttccgctcacgctcggccacgaaggcgcaggcaaggtcgccgccgtcggcgagggtgtcgaaggtctcgacatcggaaccaatgtcgtcgtctacgggccttggggttgcggcaactgttggcactgctcacaaggactcgagaactattgctctcgcgcccaagaactcggaatcaatcctcccggtctcggtgcacccggcgcgttggccgagttcatgatcgtcgattctcctcgccaccttgtcccgatcggtgacctcgacccggtcaagacggtgccgctgaccgacgccggtctgacgccgtatcacgcgatcaagcgttctctgccgaaacttcgcggaggctcgtacgcggttgtcattggtaccggcggtctcggccacgtcgctattcagctcctccgccacctctcggcggcaacggtcatcgctttggacgtgagcgcggacaagctcgaactggcaaccaaggtaggcgctcacgaagtggttctgtccgacaaggacgcggccgagaacgtccgcaagatcactggaagtcaaggcgccgcattggttctcgacttcgtcggctaccagcccaccatcgacaccgcgatggctgtcgccggcgtcggatcagacgtcacgatcgtcgggatcggggacggccaggcccacgccaaagtcgggttcttccaaagtccttacgaggcttcggtgacagttccgtattggggtgcccgcaacgagttgatcgaattgatcgacctcgcccacgccggcatcttcgacatcggcggtggagaccttcagtctcgacaacggtgccgaagcgtatcgacgactggctgccggaacgctcagcggccgtgcggttgtggtccctggtctgtagtaccgacagcggtagaacgacagcggaaaaacactgatgcccggccgaattcgattcggccgggcatcagtgtcagaaattcggtgtgcgctagctgcacgcctcgatga

SEQ ID NO: 3 - protein, 325 aa, Penicillium citrinum
MSNGKTFTLSNGVKIPGVGFGTFASEGSKGETYTAVTTALKTGYRHLDCAWYYLNEGEVGEGIRDFLKENPSVKREDIFVCTKVWNHLHRYEDVLWSIDDSLKRLGLDYVDMFLVHWPIAAEKNGQGEPKIGPDGKYVILKDLTENPEPTWRAMEKIYEDRKARSIGVSNWTIADLEKMSKFAKVMPHANQIEIHPFLPNEELVQYCFSKNIMPVAYSPLGSQNQVPTTGERVSENKTLNEIAEKGGNTLAQVLIAWGLRRGYVVLPKSSNPKRIESNFKSIELSDADFEAINAVAKGRHFRFVNMKDTFGYDVWPEETAKNLSA

SEQ ID NO: 4 - DNA, 978 bp, Penicillium citrinum
  CDS (1)..(978)
atgtctaacggaaagactttcacattgagcaacggcgtcaagattcctggcgtcggctttggtaccttcgctagtgaaggttccaagggcgagacctatactgctgtcaccactgccctgaagaccggttaccgtcacttggactgtgcctggtactacctgaacgagggtgaggttggtgagggtatccgtgacttcctgaaggagaacccctcggtgaagcgtgaggacatcttcgtctgcaccaaggtgtggaaccacctccaccgttatgaggacgtcctctggtccattgacgactccctgaagcgtcttggacttgactacgttgatatgttcctcgttcactggcccattgctgccgagaagaatggccagggtgagcccaagattggccctgacggcaaatacgtcattctcaaggacctgaccgagaaccccgagcccacatggcgcgctatggagaagatttatgaggatcgcaaggccaggtccattggtgtctccaactggaccattgccgaccttgagaagatgtccaagttcgccaaggtcatgcctcacgccaaccagatcgagattcaccccttcctgcccaacgaggagctggtgcagtactgcttctccaagaacattatgcccgtggcctactctcctctgggctcgcagaaccaggttcccaccaccggtgagcgggtcagcgagaacaagactctgaacgagatcgccgagaagggcggcaacacccttgctcaggttcttattgcctggggtctgcgccgtggctacgtcgttctccccaagagctccaaccccaagcgcattgagtccaacttcaagagcattgagctctccgatgccgactttgaagccatcaatgccgttgccaagggtcgtcacttccgtttcgtcaacatgaaggatactttcggatatgatgtctggcccgaggagaccgccaagaacctgtctgcgtga

SEQ ID NO: 5 - DNA, 27 bp, Artificial Sequence
  Description of Artificial Sequence Designed oligonucleotide primer for PCR
gccatggctatgaaggcgatccagtac

SEQ ID NO: 6 - DNA, 29 bp, Artificial Sequence
  Description of Artificial Sequence Designed oligonucleotide primer for PCR
cggatccgtcatcgaggcgtgcagctagc

SEQ ID NO: 7 - DNA, 27 bp, Artificial Sequence
  Description of Artificial Sequence Designed oligonucleotide primer for PCR
gccatggctatgtctaacggaaagact

SEQ ID NO: 8 - DNA, 29 bp, Artificial Sequence
  Description of Artificial Sequence Designed oligonucleotide primer for PCR
cggatccgttataatttcgtagagattca

SEQ ID NO: 9 - DNA, 21 bp, Artificial Sequence
  Description of Artificial Sequence Designed oligonucleotide primer for PCR
gatcatcatagcaggagtcat

SEQ ID NO: 10 - DNA, 21 bp, Artificial Sequence
  Description of Artificial Sequence Designed oligonucleotide primer for PCR
gaattcaacaccagtcagctc

SEQ ID NO: 11 - DNA, 786 bp, Bacillus megaterium
  CDS (1)..(786)
atgtataaagatttagaaggaaaagtagttgtcataacaggttcatctaccggtttaggaaaagcaatggcgattcgttttgcgacagaaaaagctaaagtagttgtgaactatcgttcgaaagaagaagaagctaacagcgttttagaagaaattaaaaaagtgggcggagaggctattgccgtcaaaggtgatgtaacagttgagtctgatgtgatcaatttagttcaatctgctattaaagaatttggaaagctagacgttatgattaataacgcaggaatggaaaatccggtttcgtctcatgaaatgtctttaagtgattggaataaagtcattgatacgaacttaacgggagcatttttaggcagccgtgaagcgattaaatattttgtggaaaatgatattaagggaacagttattaacatgtcgagtgttcacgagaaaattccttggccattatttgttcattacgcagcaagtaaaggcggaatgaagctcatgaccgaaacacttgcattagaatacgctccaaaaggtattcgtgtaaataacattggaccgggagcgattaatacaccgattaacgctgagaaatttgctgatcctgagcagcgtgcagatgtagaaagcatgattccaatgggatacattggagagccggaagaaattgcagcggttgctgcatggctagcttcttcagaggcaagttatgtaacagggattacactctttgctgacggcggtatgacacagtacccatcattccaagcaggacgcggataa

SEQ ID NO: 12 - protein, 261 aa, Bacillus megaterium
MYKDLEGKVVVITGSSTGLGKAMAIRFATEKAKVVVNYRSKEEEANSVLEEIKKVGGEAIAVKGDVTVESDVINLVQSAIKEFGKLDVMINNAGMENPVSSHEMSLSDWNKVIDTNLTGAFLGSREAIKYFVENDIKGTVINMSSVHEKIPWPLFVHYAASKGGMKLMTETLALEYAPKGIRVNNIGPGAINTPINAEKFADPEQRADVESMIPMGYIGEPEEIAAVAAWLASSEASYVTGITLFADGGMTQYPSFQAGRG

SEQ ID NO: 13 - DNA, 27 bp, Artificial Sequence
  Description of Artificial Sequence Designed oligonucleotide primer for PCR
gccatggctatgtataaagatttagaa

SEQ ID NO: 14 - DNA, 23 bp, Artificial Sequence
  Description of Artificial Sequence Designed oligonucleotide primer for PCR
cggatccgttatccgcgtcctgc

SEQ ID NO: 15 - DNA, 28 bp, Artificial Sequence
  Description of Artificial Sequence Designed oligonucleotide primer for PCR
cggatccgagcgcccaatacgcaaaccg